(19) United States
(12) Patent Application Publication
(10) Pub. No.: US 2016/0331316 A1
(43) Pub. Date: Nov. 17, 2016

(54) IMPACT PREDICTION SYSTEMS AND METHODS

(71) Applicant: ELWHA LLC, Bellevue, WA (US)

(72) Inventors: Paul G. Allen, Mercer Island, WA (US); Philip V. Bayly, St. Louis, MO (US); David L. Brody, St. Louis, MO (US); Jesse R. Cheatham, III, Seattle, WA (US); William D. Duncan, Mill Creek, WA (US); Richard G. Ellenbogen, Seattle, WA (US); Roderick A. Hyde, Redmond, WA (US); Muriel Y. Ishikawa, Livermore, CA (US); Jordin T. Kare, San Jose, CA (US); Eric C. Leuthardt, St. Louis, MO (US); Nathan P. Myhrvold, Medina, WA (US); Tony S. Pan, Bellevue, WA (US); Robert C. Petroski, Seattle, WA (US); Raul Radovitzky, Bedford, MA (US); Anthony V. Smith, Seattle, WA (US); Elizabeth A. Sweeney, Seattle, WA (US); Clarence T. Tegreene, Mercer Island, WA (US); Nicholas W. Touran, Seattle, WA (US); Lowell L. Wood, Jr., Bellevue, WA (US); Victoria Y.H. Wood, Livermore, CA (US)

(73) Assignee: ELWHA LLC, Bellevue, WA (US)

(21) Appl. No.: 14/713,853

(22) Filed: May 15, 2015

(51) Int. Cl. A61B 5/00; G01P 15/02

(52) U.S. Cl. CPC A61B 5/6802; A61B 5/6803; G01P 15/02

(57) ABSTRACT

An impact prediction system includes a processing circuit configured to receive remote tracking data from a remote tracking system located remote from a plurality of users, receive local tracking data from a plurality of local tracking devices, each local tracking device is worn by a different one of the plurality of users, and predict an impact between two or more of the plurality of users based on the remote tracking data and the local tracking data. The remote tracking data includes data regarding a location of each of the plurality of users. The local tracking data includes data regarding movement of each of the plurality of users.

### BACKGROUND

Various systems are used in applications, such as sports, motor vehicle operation, and the like, to help reduce injuries. For example, football players typically wear a football helmet and shoulder pads to minimize the risk of injury (e.g., due to collisions with other players, the ground, etc.) while playing. Similarly, motor vehicle operators such as motorcyclists often wear helmets to minimize the risk of injury (e.g., due to collisions with other motor vehicles, etc.) while driving.

### SUMMARY

One embodiment relates to an impact prediction system. The impact prediction system includes a processing circuit configured to receive remote tracking data from a remote tracking system located remote from a plurality of users, receive local tracking data from a plurality of local tracking devices, each local tracking device is worn by a different one of the plurality of users, and predict an impact between two or more of the plurality of users based on the remote tracking data and the local tracking data. The remote tracking data includes data regarding a location of each of the plurality of users. The local tracking data includes data regarding movement of each of the plurality of users.

Another embodiment relates to an impact prediction system. The impact prediction system includes an external sensor located remote from a plurality of users and configured to acquire external sensor data related to movement of the plurality of users, a plurality of user sensors, each user sensor configured to be worn by one of the plurality of users and acquire user data related to movement of the plurality of users, and a processing circuit configured to predict an impact between two or more of the plurality of users based on the external sensor data and the user data.

Another embodiment relates to an impact prediction system. The impact prediction system includes a processing circuit configured to receive location data regarding an initial location and orientation of a user from an external sensor located remote from the user, receive movement data regarding movement of the user relative to the initial location and orientation of the user from a user sensor worn by the user, and predict an impact of the user with an object based on the location data and the movement data.

Another embodiment relates to a method for predicting an impact between two or more users. The method includes receiving remote tracking data from a remote tracking system located remote from a plurality of users with a processing circuit, receiving local tracking data from a plurality of local tracking devices with the processing circuit, and predicting an impact between two or more of the plurality of users based on the remote tracking data and the local tracking data by the processing circuit. The remote tracking data includes data regarding a location of each of the plurality of users. Each local tracking device is worn by a different one of the plurality of users, and the local tracking data includes data regarding movement of each of the plurality of users.

Another embodiment relates to a method of predicting an impact. The method includes acquiring external sensor data related to movement of the plurality of users with an external sensor located remote from a plurality of users, acquiring user data related to movement of the plurality of users with a plurality of user sensors, each user sensor is configured to be worn by one of the plurality of users, and predicting an impact between two or more of the plurality of users based on the external sensor data and the user data with a processing circuit.

Another embodiment relates to a method for predicting an impact between a user and an object. The method including receiving location data regarding an initial location and orientation of the user an external sensor located remote from the user with a processing circuit, receiving movement data regarding movement of the user relative to the initial location and orientation of the user from a user sensor worn by the user with the processing circuit, and predicting an impact of the user with the object based on the location data and the movement data with the processing circuit.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a front view of a local tracking device worn by a user for an impact prediction system, according to one embodiment.

FIG. 2 is a schematic diagram of the local tracking device for the impact prediction system of FIG. 1, according to one embodiment.

FIG. 3 is an illustration of an impact prediction system with a remote tracking system and local tracking devices, according to one embodiment.

FIG. 4 is a schematic diagram of the impact prediction system of FIG. 3, according to one embodiment.

FIG. 5 is a schematic diagram of communication between a remote tracking system and local tracking systems, according to one embodiment.

FIG. 6 is a schematic diagram of communication between a remote tracking system and local tracking systems, according to another embodiment.

FIG. 7 is a block diagram of a method of predicting an impact, according to one embodiment.

FIG. 8 is a block diagram of a method of predicting an impact, according to another embodiment.

FIG. 9 is a block diagram of a method of predicting an impact, according to a third embodiment.

FIG. 10 is a block diagram of a method of recalibrating a sensor, according to one embodiment.

### DETAILED DESCRIPTION

In the following detailed description, reference is made to the accompanying drawings, which form a part thereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Referring to the Figures generally, various embodiments disclosed herein relate to an impact prediction system used to predict an impact between two or more users, one or more users and one or more objects (e.g., walls, posts, ground, trees, vehicles, etc.), or other impacts. In other embodiments, the impact prediction system may also be used to recalibrate sensors located on a local tracking device worn by a user to reduce sensor drift (e.g., when sensors provide data offset from a calibrated state, etc.). Upon detection of an impending impact, the impact prediction system may notify the local tracking device, which in turn notifies the user with an alarm (e.g., an audible notification, a visual notification, a tactile notification, etc.) via a notification device, and/or the local tracking device may activate protective equipment (e.g., selectively inflates airbags, etc.). The impact prediction system may also determine the instigator (e.g., person at fault, aggressor, etc.) involved in the impact or collision.

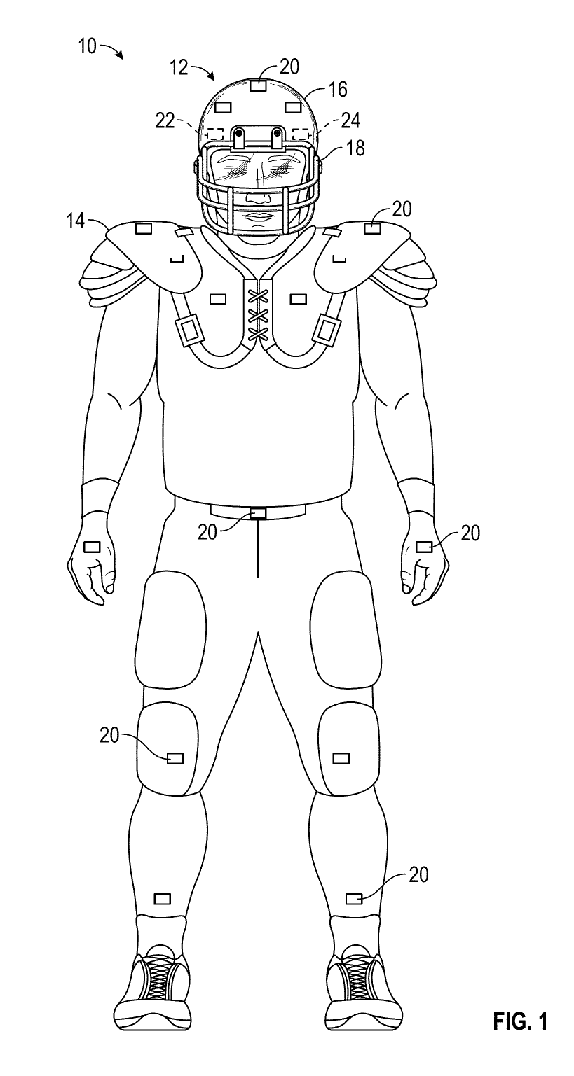

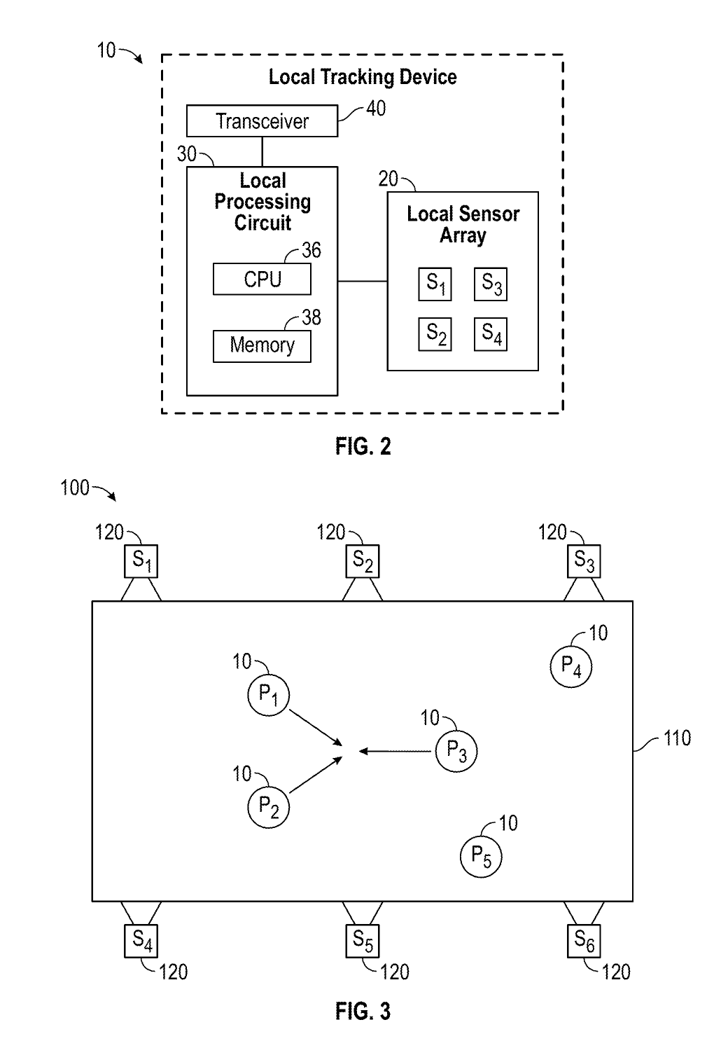

Referring to FIGS. 1-2, local tracking device 10 is shown according to one embodiment. As shown in FIG. 1, local tracking device 10 is usable to reduce the risk of injury to users while performing various activities, including playing sports (e.g., football, hockey, etc.) and operating motor vehicles (e.g., motorcycles, snowmobiles, ATVs, etc.). As shown in FIG. 1, local tracking device 10 may be coupled to helmet 12 (e.g., a head protection device or member, a first or upper protection device or member, etc.) and/or torso protection assembly 14 (e.g., a shoulder pad assembly, a second or lower protection device or assembly, etc.), and include a sensor, shown as local sensor array 20. In some embodiments, helmet 12 and torso protection assembly 14 may not be included.

In the example embodiment, helmet 12 is a football helmet. In other embodiments, helmet 12 may be any helmet used to protect a user from impacts to the head (e.g., during activities such as motocross, snowboarding, hockey, lacrosse, snowmobiling, etc.). Helmet 12 includes helmet shell 16 and facemask 18. Helmet shell 16 may be structured as any type of helmet shell (e.g., football, baseball, hockey, motocross, etc.) used to protect a user's head. Facemask 18 may be any type of helmet facemask configured to protect the user's face. In some embodiments, facemask 18 includes one or more crossbars, a transparent shield, or other protection devices. In yet further embodiments, facemask 18 is rigidly attached to helmet shell 16, forming a single continuous unitary outer shell (e.g., a motocross helmet, etc.), or removably attached (i.e., detachable) to helmet shell 16 (e.g., a hockey helmet, a football helmet, etc.). In yet further embodiments, facemask 18 is omitted (e.g., a baseball helmet, etc.).

Local sensor array 20 may be or include one or more devices (e.g., sensors, tracking devices, etc.) configured to determine the location of a user (e.g., position and/or orientation of the user and body parts relative to one another, etc.). The devices of local sensor array 20 may be positioned at various locations on the body of the user of local tracking device 10 (e.g., arms, hands, legs, feet, torso, etc.). The devices may also be disposed about helmet 12 and/or torso protection assembly 14.

In one embodiment, local sensor array 20 may determine the position and orientation of various body parts of the user and/or protective equipment (e.g., helmet 12, torso protection assembly 14, etc.). The orientation of the various body parts may include an orientation of a head, a torso, an arm, a leg, and/or any other body part. In one embodiment, one sensor or component of local sensor array 20 may act as a master device (e.g., reference location, etc.) and the sensors or components may provide their position and/or orientation relative to the master device. In other embodiments, each sensor or component may determine the position and orientation of its respective body part independent of the other sensors or components of local sensor array 20. A human body model may be used to predict the location of other body parts (e.g., body parts without a tracking device, etc.) based on the measurements (e.g., position, orientation, etc.) at each of the one or more devices of local sensor array 20.

In another embodiment, local tracking device 10 may include a beacon, shown as beacon 22. Beacon 22 may utilize radio frequency (RF), optical (e.g., infrared light (IR), etc.), and/or ultrasonic emission technologies. Beacon 22 is configured to emit signals (e.g., RF, IR, ultrasonic, etc.) that are received by external receivers/sensors (e.g., a camera device, a radar device, a lidar device, an RF receiver, etc.) to determine the position and/or orientation of the user of local tracking device 10. Beacon 22 may emit signals continuously or intermittently (e.g., based on a schedule, etc.). In some embodiments, signals from beacon 22 may include data from local tracking device 10, or local sensor array 20. In some embodiments, signals from beacon 22 may encode an identification (e.g., via frequency or pulse format of the signal, via data included in the signal, etc.) of local tracking device 10 and/or its user.

One or more devices of local sensor array 20 may include inertial navigation devices (e.g., such as an inertial navigation system (INS) including accelerometers and/or gyroscopes, etc.), cameras, sonar, and/or radar. An inertial navigation system is a navigation aid that uses a processor/computer, motion sensors (e.g., accelerometers, etc.), and rotation sensors (e.g., gyroscopes, multi-axis accelerometer arrays, etc.) to continuously or periodically calculate the position, orientation, velocity, and/or acceleration of an object, such as the user of local tracking device 10, without the need for external references. Herein, data regarding the calculated position, orientation, velocity, and/or acceleration may be referred to as local tracking data or user data.

As shown in FIG. 2, local tracking device 10 includes local processing circuit 30. Local processing circuit 30 includes local processor 36 and local memory 38. Local processor 36 may be implemented as a general-purpose processor, an application specific integrated circuit (ASIC), one or more field programmable gate arrays (FPGAs), a digital-signal-processor (DSP), a group of processing components, or other suitable electronic processing components. Local memory 38 is one or more devices (e.g., RAM, ROM, Flash Memory, hard disk storage, etc.) for storing data and/or computer code for facilitating the various processes described herein. Local memory 38 may be or include non-transient volatile memory or non-volatile memory. Local memory 38 may include database components, object code components, script components, or any other type of information structure for supporting the various activities and information structures described herein. Local memory 38 may be communicably connected to local processor 36 and provide computer code or instructions to local processor 36 for executing the processes described herein.

Referring still to FIG. 2, local sensor array 20 is communicably coupled to local processing circuit 30, such that information (e.g., positional data, orientation data, etc.) may be exchanged between local processing circuit 30 and local sensor array 20. As mentioned above, local tracking deice 10 uses a processor/computer, motion sensors (e.g., accelerometers, etc.), and rotation sensors (e.g., gyroscopes, etc.) to determine the position/location, orientation, velocity, and/or acceleration of the user. The sensors or components of local sensor array 20 (e.g., accelerometers, gyroscopes, etc.) are communicably coupled with local processing circuit 30, and more specifically, local processor 36. As such, local processor 36 receives data specific to the user of local tracking device 10 and determines the local tracking data of the user. In one embodiment, the local tracking data of the user is stored within local memory 38. In other embodiments, the local tracking data is transferred to transceiver 40 for transmission to other devices.

As shown in FIG. 2, local processing circuit 30 is communicably coupled to transceiver 40, such that information/data (e.g., local tracking data, etc.) may be exchanged between local processing circuit 30 and transceiver 40. Transceiver 40 may receive the local tracking data directly from local processor 36 and/or access the data from local memory 38. Transceiver 40 may transmit the local tracking data of the user to an external system (e.g., a remote server, a remote tracking system, an impact prediction system, etc.), as is described more fully herein. In some embodiments, beacon 22 may transmit the local tracking data of the user to an external system.

In one embodiment, transceiver 40 includes a global positioning system (GPS) receiver configured to receive absolute location data (e.g., absolute position measurements, etc.). The absolute location data may be used to reorient (e.g., recalibrate, zero out, etc.) one or more devices of local tracking device 10 (e.g., sensors, tracking devices, accelerometers, etc.) to reduce the effects of sensor drift (e.g., accelerometer drift, etc.). For example, through use of an accelerometer, the measurements may gradually begin to drift (e.g., the sensor no longer acquires accurate and precise data, etc.). Using a GPS receiver (e.g., GPS, differential GPS, augmented GPS, a GPS analog using local reference points and transmitters, etc.), local tracking device 10 may receive absolute location data to negate the effects of drift and recalibrate the device (e.g., accelerometer, etc.).

In another embodiment, the local tracking device 10 may include inclinometers and/or magnetometers configured to provide absolute location data (e.g., absolute orientation measurements, etc.) to zero out the effects of sensor drift (e.g., gyro drift of a gyroscope, etc.). The local tracking device 10 may receive the absolute tracking data (e.g., absolute orientation measurements, absolute position measurements, etc.) periodically, based on a schedule, or continuously. For example, the absolute tracking data may be received on a fixed schedule (e.g., time-based, play-based, at the start of each play in football, etc.), when the user enters area of play (e.g., field, court, track, rink, etc.), once the error covariance has degraded sufficiently (e.g., sensor drift, etc.), during a period of inactivity (e.g., during a stop in play, during a timeout, etc.) or any other appropriate time.

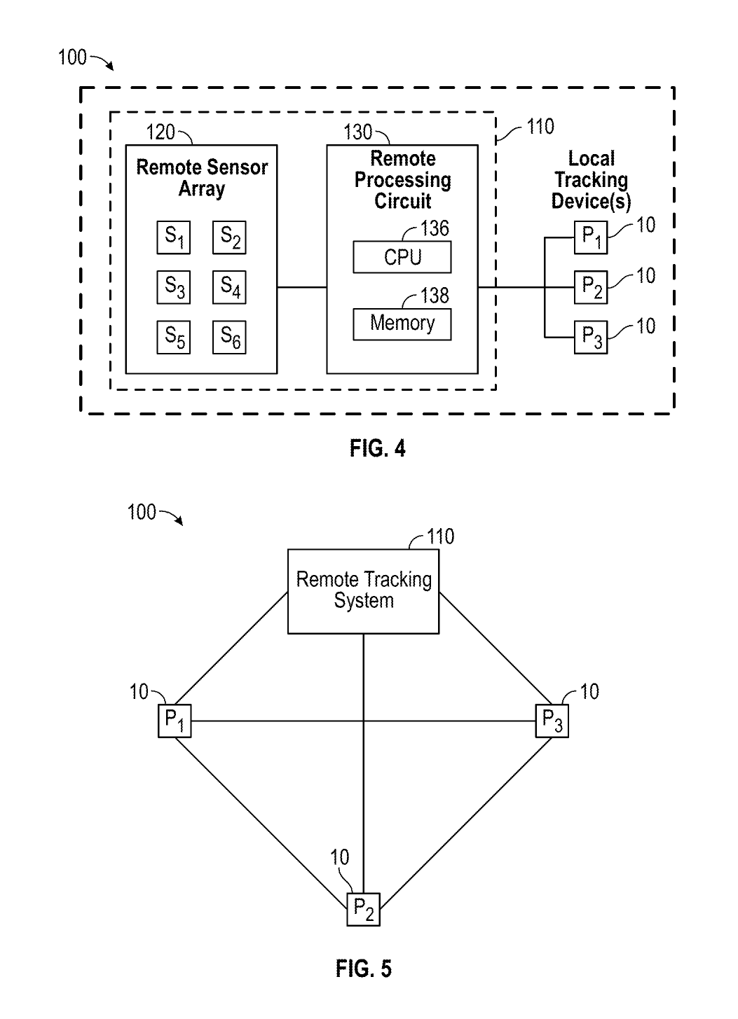

Referring now to FIGS. 3-4, an impact prediction system, shown as impact prediction system 100, is shown according to one embodiment. As shown in FIGS. 3-4, impact prediction system 100 includes one or more local tracking devices 10 (e.g., a plurality of users of local tracking devices 10, P₁, P₂, P₃, P₄, P₅, etc.) and an external tracking system, shown as remote tracking system 110. Remote tracking system 110 includes remote sensor array 120 and remote processing circuit 130. Remote sensor array 120 may be or include one or more devices (e.g., sensors, tracking devices, etc.) configured to acquire remote tracking data/signals (e.g., external sensor data, etc.) in order to continuously or periodically determine the position, orientation, velocity, and/or acceleration of each of a plurality of users (e.g., one or more users of local tracking devices 10, etc.) and/or objects. The objects may include stationary objects (e.g., the ground, walls, goalposts, a net, etc.) and/or moving objects (e.g., a vehicle, a ball, a stick, a lacrosse ball, a baseball, a puck, a hockey/lacrosse stick, a baseball bat, etc.). The one or more devices of remote sensor array 120 may include a camera device, a radar device, a lidar device, an RF receiver, and/or any other device suitable to acquire data regarding the location of each of the plurality of users and/or objects. In some embodiments, remote tracking system 1110 includes at least one of a global navigation satellite system, a global positioning system, a differential global positioning system, an augmented global positioning system, and a local positioning system and is configured to acquire remote tracking signals and/or the absolute location data.

Remote processing circuit 130 includes remote processor 136 and remote memory 138. Remote processor 136 may be implemented as a general-purpose processor, an application specific integrated circuit (ASIC), one or more field programmable gate arrays (FPGAs), a digital-signal-processor (DSP), a group of processing components, or other suitable electronic processing components. Remote memory 138 is one or more devices (e.g., RAM, ROM, Flash Memory, hard disk storage, etc.) for storing data and/or computer code for facilitating the various processes described herein. Remote memory 138 may be or include non-transient volatile memory or non-volatile memory. Remote memory 138 may include database components, object code components, script components, or any other type of information structure for supporting the various activities and information structures described herein. Remote memory 138 may be communicably connected to remote processor 136 and provide computer code or instructions to remote processor 136 for executing the processes described herein.

As shown in FIG. 4, remote sensor array 120 is communicably coupled to remote processing circuit 130, such that information (e.g., remote tracking data, etc.) may be exchanged between remote processing circuit 130 and remote sensor array 120. The remote tracking data may be stored in remote memory 138. The one or more local tracking devices 10 are communicably coupled to remote processing circuit 130 of remote tracking system 110 via transceivers 40 (see FIG. 2) and/or beacons 22, such that information (e.g., local tracking data, etc.) may be exchanged between remote processing circuit 130 and each of the one or more local tracking devices 10. The local tracking data provides an indication of an acceleration, a velocity, a location, and/or an orientation for each of the plurality of users. The local tracking data received by remote processing circuit 130 may be stored in remote memory 138.

In one embodiment, remote processor 136 accesses remote memory 138 to compare the local tracking data and the remote tracking data. By comparing the local tracking data and the remote tracking data, remote processing circuit 130 may determine an amount of drift for each of the plurality of local tracking devices 10. Thereby, remote tracking system 110 may reduce drift associated with the local tracking data by providing absolute location data to each local tracking device 10 to recalibrate (i.e., zero out, etc.) the one or more sensors (e.g., accelerometers, gyroscopes, etc.) of each local tracking device 10. In one embodiment, remote tracking system 110 determines the absolute location data via a camera device, a radar device, and/or a lidar device. In another embodiment, remote tracking system 110 determines the absolute location data from receiving signals from user-mounted beacons (e.g., beacons 22, etc.). In one embodiment, remote tracking system 110 includes multiple signal receivers located at different sites, which may respectively receive signals from beacons 22 and determine the absolute tracking information via triangulation. In another embodiment, remote tracking system 110 includes multiple signal receivers located at different sites, which may respectively receive signals from beacons 22 and determine the absolute tracking information via comparing range information (e.g., determined from signal transit times, from signal intensities, etc.). In other embodiments, remote tracking system 110 includes at least one ranging and imaging signal receiver, which may receive signals from beacons 22 and determine the absolute tracking information based on direction and range. Beacons 22 may transmit signals on an intermittent or continuous basis. An intermittent beacon may be activated based on a schedule (e.g., one player at a time, etc.) and/or based on a query by remote tracking system 110 (e.g., remote tracking system 110 asks about an individual local tracking device 10 when information is needed on the individual local tracking device 10, etc.).

Remote tracking system 110 may determine the absolute tracking data periodically, based on a schedule, or continuously. In one embodiment, local tracking devices 10 receive the absolute tracking data quasi-synchronously (i.e., all at the same time, simultaneously, etc.). For example, in football, each local tracking device 10 receives the absolute tracking data at the start of each play, during a time-out, or other breaks in play where the users of local tracking devices 10 are substantially inactive (e.g., standing still, etc.). However, the absolute tracking data may be determined during a period of activity (e.g., while a user is moving, etc.). In another embodiment, local tracking devices 10 receive the absolute tracking data asynchronously. For example, individual local tracking devices 10 receive the absolute tracking data on a fixed schedule (e.g., when the user enters the area of play, once the error covariance degrades sufficiently, based on a length of time in play, etc.) independent of when other local tracking devices 10 receive the absolute tracking data.

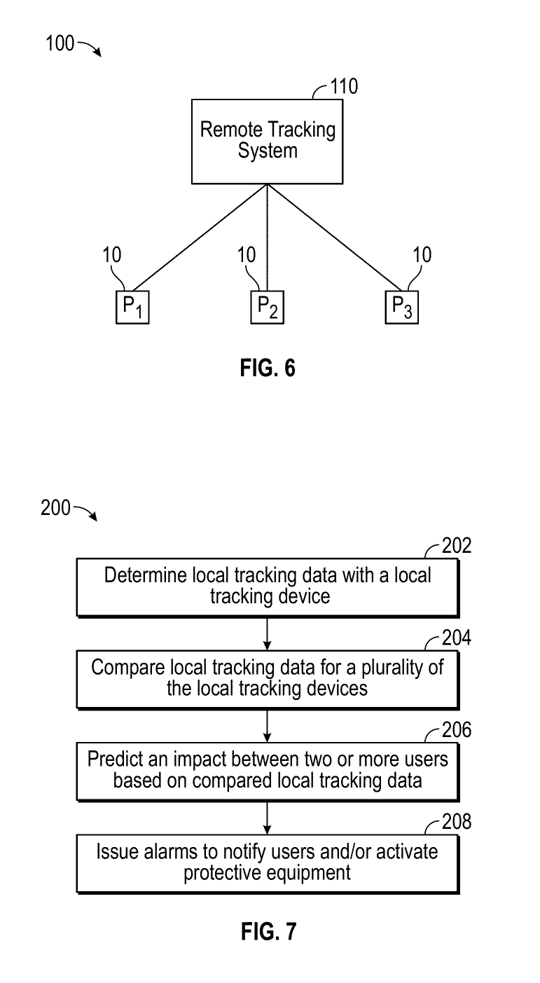

Referring to FIGS. 5-6, the impact prediction system 100 may use one or both of the local tracking devices 10 and the remote tracking system 110 to predict an impact between two or more users, a user and an object (e.g., wall, ground, post, ball, stick, etc.), or other collisions. According to the example embodiment shown in FIG. 5, local tracking devices 10 may communicate with one another to compare local tracking data (e.g., position, orientation, velocity, acceleration, etc.). By way of example, by comparing the local tracking data, the various local processing circuits 30 may determine at least one of current separations, relative (e.g., differential, etc.) velocities, and relative accelerations between two or more users. In one embodiment, the comparison of local tracking data is performed between two users of local tracking devices 10. For example, the comparison of the local tracking data between a first local tracking device 10 (e.g., P₁, etc.) and a second local tracking device 10 (P₂, etc.) is used to determine the current separation, the relative velocity, and/or the relative acceleration between the first and second local tracking devices. In other embodiments, the local tracking data for three or more users is compared (e.g., P₁, P₂, P₃, etc.). In another embodiment, local tracking data for a user of one of the local tracking devices is compared to the location (e.g., relative location, etc.) and/or movement of one or more objects (e.g., walls, ground, posts, balls, sticks, etc.).

Using the compared local tracking data, local tracking devices 10 via local processing circuits 30 may predict whether two or more users, a user and an object, or one or more users and one or more objects are likely to collide. The collision predictions may include predictions of closing velocity (e.g., the relative velocity of the impacting bodies at the time of collision, etc.), impact locations (e.g., a user's head, torso, leg, etc.), directions of each impacting user relative to each other or to their head direction, impact time, impact severity (e.g., based on closing speed and impact location), and/or any other pertinent collision characteristics (e.g., impact parameters, etc.).

In another embodiment, both local tracking devices 10 and remote tracking system 110 may independently predict an impact between two or more users (or objects). As shown in FIG. 5, local tracking devices 10 may communicate with one another and remote tracking system 110 to predict a collision between two or more users. For example, local tracking devices 10 may communicate with one another as described above to determine at least one of current separations, relative velocities, and relative accelerations between two or more users to predict an impact between the two or more users. Remote tracking system 110 may compare remote tracking data for each of the plurality of users (e.g., P₁, P₂, P₃, etc.) to determine at least one of current separations, relative velocities, and relative accelerations between two or more users (e.g., without receiving local tracking data, etc.) to predict an impact between the two or more users. The impact predictions of local tracking devices 10 may be received by remote tracking system 110 and compared to the impact predictions of remote tracking system 110 (e.g., via remote processing circuit 130, etc.). The comparison between the two impact predictions may allow for a more precise and accurate determination of the impact parameters (e.g., time until impact, impacting force, impacting velocity, impact location, etc.).

In another embodiment, the impact prediction system 10 may use the location and/or movement of one or more moving objects to predict how and where a player (or players) may move (e.g., in response to a ball or puck being put into play and in relation to the player(s), etc.) to predict collisions. For example, in a football game, the movement and/or final location of a wide receiver and a defensive back may be predicted by tracking the trajectory of a football (e.g., during a pass play, etc.). Additionally or alternatively, the impact prediction system 10 may use the location and/or movement of stationary or semi-stationary objects, such as a net (e.g., a hockey net, etc.), to predict how a player (or players) may move (e.g., to avoid the object, etc.) or alter their movement/trajectory as they come into contact with the object. For example, in a hockey game, a player on an offensive attack may approach the net at an angle and speed that requires them to cut quickly around the front or back of the net to avoid a collision with the goalie. Therefore, the impact prediction system 10 may interpret this to predict a potential collision between the offensive player and a defensive player around the net.

According to the example embodiment shown in FIG. 6, local tracking devices 10 may communicate with remote tracking system 110 to compare local tracking data and remote tracking data for two or more users of local tracking devices 10. Remote tracking system 110 via remote processing circuit 130 may then predict an impact (e.g., closing velocity, impact locations, directions of each impacting user relative to each other or to their head direction, impact time, impact severity, etc.) between two or more of the plurality of users (e.g., P₁, P₂, P₃, etc.) based on the local tracking data and the remote tracking data.

In an alternative embodiment, remote tracking system 110 receives location data regarding an initial location and orientation of a user and/or an object from at least one of remote sensor array 120 and local sensor array 20. Remote tracking system 110 also receives movement data regarding movement of the user and/or object relative to the initial location and orientation of the user and/or object from at least one of remote sensor array 120 and local sensor array 20. Remote processing circuit 130 then predicts an impact of the user with the object (e.g., wall, ground, post, ball, stick, etc.) based on the location data and the movement data.

In another embodiment, remote tracking system 110 may compare remote tracking data for each of the plurality of users (e.g., P₁, P₂, P₃, etc.) to determine at least one of current separations, relative velocities, and relative accelerations between two or more users and/or objects (e.g., without receiving local tracking data, etc.). Using the remote tracking data for each of the plurality of users, remote tracking system 110 may predict whether one or more users and/or objects are likely to collide. The collision predictions may include predictions of closing velocity, impact locations, directions of each impacting user relative to each other or to their head direction, impact time, impact severity, and/or any other pertinent collision characteristics.

The various embodiments of predicting an impact described above may be used to notify one or more users involved in the potential collision. In one embodiment, the collision prediction is used to issue an alarm. For example, a notification device, shown as notification device 24, of local tracking device 10 may be configured to convey the alarm (e.g., audible indicator, vibratory tactile feedback, visual indicator, etc.) to a user to notify the user of the impending impact. By notifying the user, the user may be able to avoid the collision or brace themselves for the impending impact. The alarms may be conditional based on the predicted severity or magnitude of the impact. For example, sub-threshold impacts (e.g., small impacts, non-severe impacts, etc.) may not set of an alarm or may trigger a different type of alarm than an impact exceeding an impact threshold (e.g., a target force, a target velocity, etc.). The alarms may include details about the collision (e.g., different types of alarms convey different impact parameters, etc.). For example, the alarms may convey details about a potential collision such as an expected severity (e.g., closing speed, impulse, etc.), an impact location (e.g., head, torso, legs, etc.), the relative direction (e.g., lateral, longitudinal, front, rear, side, etc.), the nature of the impacting object (e.g., a helmet, a knee, an arm, a wall, a post, a ball, a stick, etc.), time until impact, and/or other impact parameters. Alarm thresholds may be customized on an individual basis such that alarms may be selectively provided on a relatively more or less conservative basis.

In another embodiment, the impact prediction is used to activate protective equipment in order to negate or substantially reduce the magnitude of the impact in order to, among other things, minimize accelerations experienced by the head and neck portions or other areas of the user and reduce the risk of the user experiencing a concussion or other undesirable injuries. For example, upon detection of an impending impact, local tracking device 10 may intelligently (e.g., selectively, etc.) inflate various airbags from helmet 12 or other locations on or within local tracking device 10 to minimize forces and torques on its wearer. In some embodiments, local tracking device 10 may actively inflate or deflate one or more airbags before and/or during a collision. In other embodiments, local tracking device 10 may communicate with one or more other local tracking devices 10 to determine a course of action regarding inflation of airbags of each local tracking device 10 in an impending impact. In further embodiments, local tracking device 10 may inflate an airbag to resist relative movement between helmet 12 and torso protection assembly 14 to reduce risk of injury to the user. For example, the airbag may couple helmet 12 and torso protection assembly 14 to prevent or resist relative movement between the two.

According to an example embodiment, remote tracking system 110 and each of the plurality of local tracking devices 10 may work individually or in unison to identify the users involved in the collision. In one embodiment, identifying the users in the collision may help support staff (e.g., trainers, doctors, coaches, etc.) maintain appropriate medical attention with users who may have been involved in a substantial impact, potentially leading to an injury (e.g., concussion, etc.). In other embodiments, the impact prediction system 100 may predict or determine who the instigator (e.g., person at fault, aggressor, etc.) is in the collision. Determining the instigator in the collision may be based on the location of the impact on each player, the velocity of each player, the acceleration of each player, and/or still other characteristics. For example, if a collision between two players results in an impact to the side or back of a first player's head, the second player is most likely the instigator. Identifying the instigator in the collision may help officials (e.g., referees, umpires, sirs, league administration, etc.) take appropriate action such as fining, suspending, penalizing, and/or taking other appropriate action against the instigator.

Referring now to FIG. 7, method 200 of predicting an impact is shown according to an example embodiment. In one example embodiment, method 200 may be implemented with local tracking devices 10 of FIGS. 1-4. Accordingly, method 200 may be described in regard to FIGS. 1-4.

At 202, local tracking data is determined using a local tracking device. For example, local tracking device 10 may use local senor array 20 to continuously or periodically determine the position, orientation, velocity, and/or acceleration of an object, such as the user of local tracking device 10. At 204, the local tracking data for a plurality of local tracking devices is compared. For example, via transceivers 40, local tracking devices 10 may compare the local tracking data with each of the other local tracking devices 10 in the system (e.g., on the field, in play, etc.). The compared local tracking data may allow the local tracking devices 10 to determine current separations, relative velocities, and relative accelerations between two or more users (e.g., via local processing circuits 30, etc.). At 206, an impact between two or more users is predicted based on the compared local tracking data. For example, a first local tracking device 10 (e.g., P₁, etc.) may predict that it is about to be involved in a collision between one or more other local tracking devices 10 (e.g., P₂, P₃, etc.).

At 208, alarms are issued to notify the users and/or the users protective equipment is activated. For example, in one embodiment, the individual local tracking devices 10 may notify its user of the impending impact via an alarm (e.g., such as an audible indicator, vibratory tactile feedback, a visual indicator, etc.) conveyed by the notification device 24. In another embodiment, local tracking devices 10 may inflate various airbags and/or activate other protection equipment to reduce the magnitude of the impact on the user. In some embodiments, the local tracking devices 10 may both issue an alarm and activate protective equipment.

Method 200 is shown to only encompass users of local tracking devices 10. In one embodiment, method 200 may involve a local tracking device 10 and potential/actual impacts with the ground or other object (e.g., a wall, a post, a tree, a vehicle, a ball, a stick, etc.). In other embodiments, method 200 may involve any plurality of user of local tracking devices 10 and any plurality of objects.

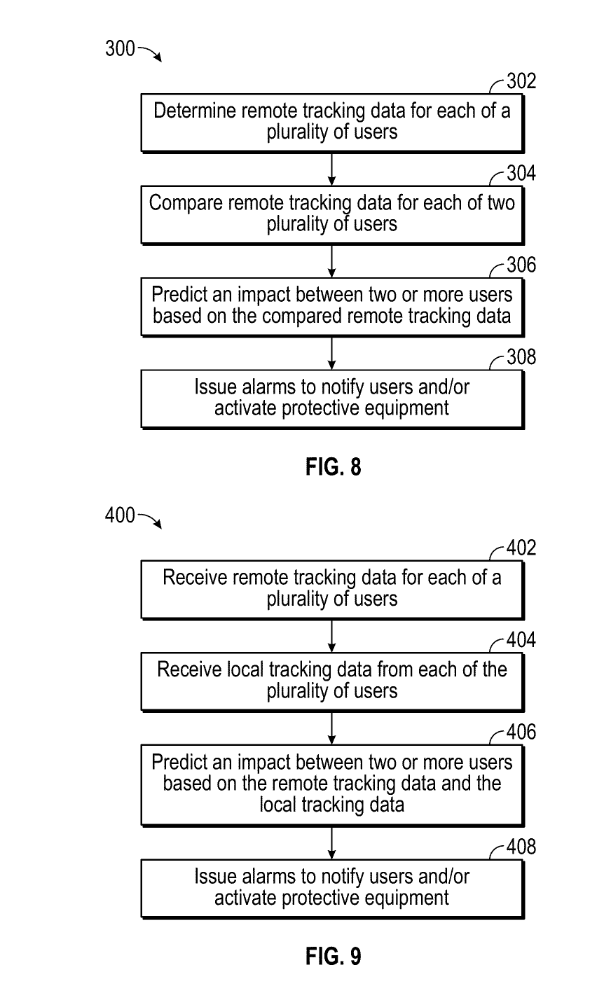

Referring now to FIG. 8, method 300 of predicting an impact is shown according to an example embodiment. In one example embodiment, method 300 may be implemented with remote tracking system 110 of FIGS. 3-4. Accordingly, method 300 may be described in regard to FIGS. 3-4.

At 302, remote tracking data is determined for each of a plurality of users of local tracking devices. For example, remote tracking system 110 may use remote senor array 120 to continuously or periodically determine the position, orientation, velocity, and/or acceleration of a plurality of objects, such as the users of local tracking devices 10 (e.g., P₁, P₂, P₃, etc.). At 304, the remote tracking data for each of the plurality of users of local tracking devices is compared. For example, remote processing circuit 130 may compare the remote tracking data for each local tracking device 10 in the system (e.g., on the field, in play, etc.). The compared remote tracking data may allow the remote processing circuit 130 to determine current separations, relative velocities, and relative accelerations between two or more users of the local tracking devices 10. At 206, an impact between two or more users is predicted based on the compared remote tracking data. For example, remote tracking system 110 may predict that a first user of a local tracking device 10 (e.g., P₁, etc.) is about to be involved in a collision between one or more other users of local tracking devices 10 (e.g., P₂, P₃, etc.).

At 308, alarms are issued to notify the users and/or the users protective equipment is activated. For example, in one embodiment, remote tracking system 110 may communicate with individual local tracking devices 10 to notify its user of the impending impact via an alarm (e.g., such as an audible indicator, vibratory tactile feedback, a visual indicator, etc.) conveyed by the notification device 24. In another embodiment, remote tracking system 110 may communicate with individual local tracking devices 10 to inflate various airbags and/or activate other protection equipment to reduce the magnitude of the impact on the user. In some embodiments, remote tracking system 110 may communicate with individual local tracking devices 10 to both issue an alarm and activate protective equipment.

Method 300 is shown to encompass only users of local tracking devices 10 being monitored by remote tracking system 110. In one embodiment, method 300 may involve a local tracking device 10 and potential/actual impacts with the ground or other object (e.g., a wall, a post, a tree, a vehicle, a ball, a stick, etc.). In other embodiments, method 300 may involve any plurality of user of local tracking devices 10 and any plurality of objects.

Referring now to FIG. 9, method 400 of predicting an impact is shown according to an example embodiment. In one example embodiment, method 400 may be implemented with impact prediction system 100 of FIGS. 3-4. Accordingly, method 400 may be described in regard to FIGS. 3-4.

At 402, remote tracking data is received by impact prediction system 100 for each of a plurality of users of local tracking devices 10. For example, remote tracking system 110 may use remote senor array 120 to continuously or periodically determine the position, orientation, velocity, and/or acceleration of a plurality of objects, such as the users of local tracking devices 10 (e.g., P₁, P₂, P₃, etc.). At 404, local tracking data is received by impact prediction system 100 for each of the plurality of users of local tracking devices 10. For example, local tracking devices 10 may use local senor arrays 20 to continuously or periodically determine the position, orientation, velocity, and/or acceleration of the users of local tracking devices 10. The local tracking data may be sent to impact prediction system 100 from transceivers 40 of local tracking devices 10 to remote processing circuit 130. At 406, an impact between two or more users is predicted based on the remote tracking data and the local tracking data. For example, impact prediction system 100 may compare the remote tracking data and the local tracking data. Based on the compared remote tracking data and local tracking data, remote processing circuit 130 may predict an impact (e.g., closing velocity, impact locations, directions of each impacting user relative to each other or to their head direction, etc.) between two or more of the plurality of users (e.g., P₁, P₂, P₃, etc.).

At 408, alarms are issued to notify the users and/or the users protective equipment is activated. For example, in one embodiment, remote tracking system 110 may communicate with individual local tracking devices 10 to notify its user of the impending impact via an alarm (e.g., such as an audible indicator, vibratory tactile feedback, a visual indicator, etc.) conveyed by the notification device 24. In another embodiment, remote tracking system 110 may communicate with individual local tracking devices 10 to inflate various airbags and/or activate other protection equipment to reduce the magnitude of the impact on the user. In some embodiments, remote tracking system 110 may communicate with individual local tracking devices 10 to both issue an alarm and activate protective equipment.

Method 400 is shown to only encompass users of local tracking devices 10. In one embodiment, method 400 may involve a local tracking device 10 and potential/actual impacts with the ground or other object (e.g., a wall, a post, a tree, a vehicle, a ball, a stick, etc.). In other embodiments, method 400 may involve any plurality of user of local tracking devices 10 and any plurality of objects.

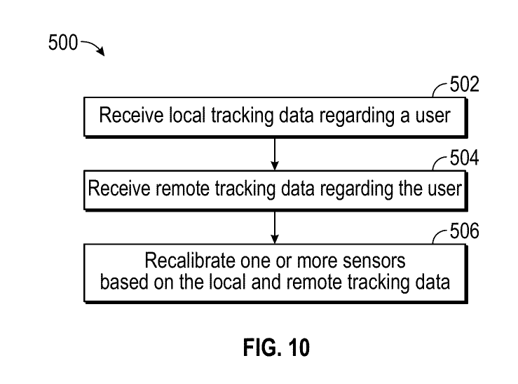

Referring now to FIG. 10, method 500 of recalibrating one or more sensors is shown according to an example embodiment. In one example embodiment, method 500 may be implemented with local tracking device 10 of FIGS. 1-2. Accordingly, method 500 may be described in regard to FIGS. 1-2. In another example embodiment, method 500 may be implemented with local tracking device 10 and remote tracking system 110 of FIGS. 3-4. Accordingly, method 500 may be described in regard to FIGS. 3-4.

At 502, local tracking data is received by remote tracking device 110. For example, at the start of a play, local tracking device 10 may determine position, orientation, velocity, and/or acceleration regarding a user via local sensor array 20. At 504, remote tracking data is received by remote tracking device 110 by remote sensor array 120. In one embodiment, the remote tracking data is determined at the exact same or substantially the same place and time as the local tracking data.

At 506, one or more sensors of local tracking device 10 are recalibrated based on the local and remote tracking data. For example, by comparing the local tracking data and the remote tracking data, remote processing circuit 130 may determine an amount of drift for local tracking device 10. Thereby, remote tracking system 110 may reduce drift associated with the local tracking data by providing absolute location data to each local tracking device 10 to recalibrate the sensors. In an alternative embodiment of method 500, local tracking device 10 may include a GPS receiver configured to receive absolute location data. The absolute location data may be used to recalibrate one or more devices of local tracking device 10 to reduce the effects of sensor drift. Method 500 is shown to include a single user of local tracking device 10. In one embodiment, method 500 may involve a plurality of user of local tracking devices 10.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general purpose computer, special purpose computer, or special purpose processing machines to perform a certain function or group of functions.

Although the figures may show a specific order of method steps, the order of the steps may differ from what is depicted. Also two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. An impact prediction system, comprising:
a notification device worn by one of a plurality of users, wherein the notification device is configured to activate protective equipment worn by the one of the plurality of users; and
a processing circuit configured to:
receive remote tracking data from a remote tracking system located remote from the plurality of users, wherein the remote tracking data includes information associated with a location of each of the plurality of users;
receive local tracking data from a local sensor, wherein the local tracking data includes data regarding movement of at least the one of the plurality of users;
predict an impact between two or more of the plurality of users based on the remote tracking data and the local tracking data; and
control operation of the notification device based on the predicted impact between the two or more of the plurality of users.

2. The system of claim 1, wherein the local tracking data includes an indication of at least one of an acceleration, a velocity, and a location of the at least the one of the plurality of users.

3. The system of claim 1, wherein the local tracking data includes an indication of an orientation of the at least the one of the plurality of users.

4. (canceled)

5. The system of claim 1, wherein the processing circuit is configured to compare the information from the remote tracking data and the local tracking data to reduce drift associated with the local tracking data.

6. The system of claim 1, further comprising the remote tracking system, wherein the remote tracking system includes at least one of a camera device, a radar device, a lidar device, and an RF receiver.

7-13. (canceled)

14. The system of claim 1, further comprising the local sensor, wherein the local sensor is configured to acquire the local tracking data that includes information regarding movement of at least the one of the plurality of users wherein the sensor includes at least one of an accelerometer and a gyroscope.

15. The system of claim 14, wherein the processing circuit is configured to receive absolute location data regarding each of the plurality of users to reduce drift associated with the local sensor.

16-17. (canceled)

18. The system of claim 1, wherein the information from the remote tracking data further includes an indication of at least one of an acceleration, a velocity, and an orientation of each of the plurality of users.

19-24. (canceled)

25. The system of claim 1, wherein the remote tracking data further includes information regarding at least one of a location and a movement of an inanimate object.

26. The system of claim 25, wherein the inanimate object includes a moving object.

27. The system of claim 25, wherein the processing circuit is configured to predict an impact between one or more of the plurality of users and the inanimate object based on the information from the remote tracking data and the local tracking data.

28-30. (canceled)

31. An impact prediction system, comprising:
a plurality of user devices, each user device configured to be worn by one of a plurality of users, each user device including:
a user sensor configured to acquire user data related to movement of at least the one of the plurality of users; and
a notification device configured to activate protective equipment; and
an external tracking system located remote from the plurality of users, the external tracking system including:
an external sensor configured to acquire external sensor data related to at least one of movement and a location of each of the plurality of users; and
a processing circuit configured to receive the user data from each of the plurality of user devices, receive the external sensor data from the external sensor, predict an impact between two or more of the plurality of users based on the external sensor data and the user data, and control operation of the notification device of the two or more of the plurality of users based on the predicted impact between the two or more of the plurality of users.

32. The system of claim 31, wherein the user data includes an indication of at least one of an acceleration, a velocity, and a location of the at least the one of the plurality of users.

33. The system of claim 31, wherein the user data includes an indication of an orientation of the at least the one of the plurality of users.

34. (canceled)

35. The system of claim 31, wherein the processing circuit is configured to compare the external sensor data and the user data to reduce drift associated with the user sensors.

36. The system of claim 31, wherein the external sensor includes at least one of a camera device, a radar device, a lidar device, and an RF receiver.

37-47. (canceled)

48. The system of claim 31, wherein the external sensor data includes an indication of at least one of an acceleration, a velocity, a position, and an orientation of each of the plurality of users.

49-54. (canceled)

55. The system of claim 31, wherein the external sensor data further includes data regarding at least one of a location and a movement of an inanimate object.

56. (canceled)

57. The system of claim 55, wherein the processing circuit is configured to predict an impact between one or more of the plurality of users and the inanimate object based on the external sensor data and the user data.

58. The system of claim 57, wherein the processing circuit is configured to control operation of the notification device of the one or more of the plurality of users based on the predicted impact between the one or more of the plurality of users and the inanimate object.

59-60. (canceled)

61. An impact prediction system, comprising:
a remote processing circuit located remote from a user, the remote processing circuit configured to:
receive a signal associated with location data regarding an initial location and orientation of the user from an external sensor located remote from the user;
receive a signal associated with movement data regarding movement of the user relative to the initial location and orientation of the user from a user sensor worn by the user; and
predict an impact of the user with an object based on the location data and the movement data; and
a local processing circuit positioned on the user and communicably coupled to the remote processing circuit, the local processing circuit configured to control operation of a notification device worn by the user to activate protective equipment based on the predicted impact between the user and the object.

62. The system of claim 61, wherein the movement data includes an indication of at least one of an acceleration, a velocity, an orientation, and a location of the user relative to the initial location and orientation.

63. The system of claim 62, wherein the movement data includes an indication of at least one of an acceleration, a velocity, an orientation, and a location of the user relative to the object.

64. (canceled)

65. The system of claim 61, further comprising the external sensor, wherein the external sensor includes at least one of a camera device, a radar device, a lidar device, and an RF receiver.

66-67. (canceled)

68. The system of claim 61, wherein the user sensor includes at least one of a beacon, a transmitter, and a transceiver configured to emit the signal associated with the movement data.

69-75. (canceled)

76. The system of claim 61, further comprising the notification device and the user sensor.

77. The system of claim 61, wherein the remote processing circuit is configured to provide a the notification to the local processing circuit based on the predicted impact between the user and the object.

78-80. (canceled)

81. The system of claim 61, wherein at least one of the location data and the movement data further includes data regarding the object.

82-170. (canceled)

171. The system of claim 1, wherein the notification device is further configured provide a notification to the one of the plurality of users, wherein the notification includes at least one of a tactile notification, an audible notification, and a visual notification.

172. The system of claim 171, wherein the notification comprises information regarding at least one of a predicted impact time, a predicted impact severity, a predicted impact direction, and a predicted impact location.

173. The system of claim 27, wherein the processing circuit is configured to control operation of the notification device to at least one of (i) provide a notification through a tactile notification to the one of the plurality of users regarding the predicted impact between the one or more of the plurality of users and the inanimate object and (ii) activate the protective equipment based on the predicted impact between the one or more of the plurality of users and the inanimate object.

174. The system of claim 31, wherein the notification device is further configured provide a notification to the one of the plurality of users, and wherein the notification comprises information regarding at least one of a predicted impact time, a predicted impact severity, a predicted impact direction, and a predicted impact location.

175. The system of claim 174, wherein the notification includes at least one of a tactile notification, an audible notification and a visual notification.

176. The system of claim 61, wherein the local processing circuit is further configured to control operation of the notification device worn by the user to provide a notification to the one of the plurality of users, wherein the notification comprises information regarding at least one of a predicted impact time, a predicted impact severity, a predicted impact direction, and a predicted impact location.

177. The system of claim 61, wherein the object includes at least one of an inanimate object and another user.
